(19) United States
(12) Patent Application Publication
(10) Pub. No.: US 2006/0105045 A1
(43) Pub. Date: May 18, 2006

(54) Cyclodextrin solubilizers for liquid and semi-solid formulations

(76) Inventors: Charles Michael Buchanan, Kingsport, TN (US); Norma Lindsey Buchanan, Kingsport, TN (US); Juanelle Little Lambert, Gray, TN (US); Jessica Dee Posey-Dowty, Kingsport, TN (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/268,630

(22) Filed: Nov. 8, 2005

Related U.S. Application Data
(60) Provisional application No. 60/626,005, filed on Nov. 8, 2004.

(51) Int. Cl. A61K 31/724; A61K 9/14

(52) U.S. Cl. 424/486; 514/58

(57) ABSTRACT

This invention is directed to compositions comprising a water-miscible organic solvent or solvent mixture and one or more cyclodextrin derivatives soluble in the organic water-miscible organic solvent or solvent mixture. In some embodiments, the composition comprises one or more compounds. The invention is further directed to capsules containing the compositions of the present invention and the administration of pharmaceutically active compounds or molecules to subjects. The invention also includes methods of enhancing the solubility of a compound comprising forming a complex or mixture of a compound with a hydroxybutenyl cyclodextrin and a water-miscible solvent.

This application claims the benefit of priority of U.S. Provisional Patent Application No. 60/626,005, filed Nov. 8, 2004.

Cyclodextrins (CDs) are cyclic oligomers of glucose, which typically contain 6, 7, or 8 glucose monomers joined by α-1,4 linkages. These oligomers are commonly called α-CD, β-CD, and γ-CD, respectively. Higher oligomers containing up to 12 glucose monomers are also known. Topologically, CDs can be represented as a toroid in which the primary hydroxyls are located on the smaller circumference, and the secondary hydroxyls are located on the larger circumference. Because of this arrangement, the interior of the torus is hydrophobic while the exterior is sufficiently hydrophilic to allow the CD to be dissolved in water. This difference between the interior and exterior faces allows the CD to act as a host molecule and to form inclusion complexes with guest molecules, provided that the guest molecule is of the proper size to fit in the cavity. The CD inclusion complex can then be dissolved in water thereby providing for the introduction of guest molecule that has little or no aqueous solubility into an aqueous environment. Reviews of CD complexes can be found in Chem. Rev., 1997, 97, 1325-1357 and in Supramolecular Chemistry, 1995, 6, 217-223.

Unmodified cyclodextrins, especially β-cyclodextrin, have limited aqueous solubility, have relative large molecular weights, and tend to crystallize from solution. The combination of these issues means that their ability to solubilize and stabilize guest molecules in an aqueous environment is limited. Additionally, unmodified cyclodextrins, e.g. β-cyclodextrin, have been shown to cause renal and liver damage after parenteral administration. These issues have led to exploration of the use of chemically modified or derivatized cyclodextrins that avoid some of these problems. Two examples of derivatized cyclodextrins are hydroxybutenyl cyclodextrins (HBenCD), which are disclosed in U.S. Pat. No. 6,479,467 (2002) and in Carbohydrate Research, 2002, 327(6), 493-507, and sulfonated hydroxybutenyl cyclodextrins (SulfoHBenCD), which are disclosed in U.S. Pat. No. 6,610,671.

Because of the favorable properties of modified CDs, it would be desirable to find means for using them to enhance the solubility of lipophilic or hydrophobic compounds in an aqueous physical environment. Such compositions would have many uses. One such use would be in preparation of pharmaceutical compositions. Many commercially available oral pharmaceutical formulations are sold as capsules containing non-aqueous based liquid or semi-solid water-soluble formulations. Many capsule formulations contain the active ingredients and other excipients in pharmaceutically acceptable water-miscible organic solvents such as polyethylene glycol 300 and 400 (PEG 300 and PEG 400), ethanol, propylene glycol, and glycerin in the presence of limited amounts or absence of water. However, unmodified CDs and most modified CDs have inadequate solubility in water-miscible organic solvents. Hence, efforts to resolve these problems have focused on enhancing solubility through the use of oils, surfactants, and fats, some of which raise problems of their own by destabilizing the formulation or causing undesirable side effects.

Finding a workable means for using CD derivatives to develop pharmaceutical formulations in nonaqueous solvents has proven challenging. Many CDs and CD derivatives form inclusion complexes with desirable solvents such as ethanol, propylene glycol, and polyethylene glycol. Thus, organic solvents such as polyethylene glycol would be expected to compete with a desired guest molecule for binding with a cyclodextrin or cyclodextrin derivative and thereby decrease the ability of the cyclodextrin and cyclodextrin derivative to enhance the solubility of the desired guest compound. Some surfactants and emulsifying agents are competitive and inhibitory to desired inclusion complex formation.

Thus, there is a need in the art for cyclodextrins or cyclodextrin derivatives that can be used as solubility enhancers when formulated in water-miscible organic solvents. There is also a need for compositions that contain the CDs and/or CD derivatives along with the water-miscible organic solvents, and a need for such compositions that further contain desired guest molecules. One non-limiting example of a desired guest molecule would be a lipophilic compound having pharmaceutical uses.

### SUMMARY OF THE INVENTION

This invention is directed to compositions comprising a water-miscible organic solvent, a cyclodextrin derivative and a compound, wherein the composition contains 10 wt % or less water.

In another aspect, this invention is directed to compositions comprising a water-miscible organic solvent, a cyclodextrin derivative and a compound, wherein the water-miscible organic solvent contains 10 wt % or less water.

The present invention also relates to methods of enhancing the solubility of a compound in aqueous media comprising forming a mixture or complex between the compound and a cyclodextrin derivative in a water-miscible organic solvent, wherein the mixture or complex contains 10 wt % or less water.

In another aspect, the invention relates to methods of increasing the bioavailability of a compound comprising administering the compositions of the invention to a subject such as a human.

Additional aspects and advantages of the invention will be set forth in part in the description which follows, and in part will be obvious from the description, or may be learned by practice of the invention. The embodiments and advantages of the invention can be realized and attained by means of the elements and combinations particularly pointed out in the appended claims.

It is to be understood that both the foregoing general description and the following detailed description are exemplary and explanatory only and are not restrictive of the invention, as claimed. The accompanying drawings, which are incorporated in and constitute part of this specification, and together with the description, serve to explain principles of the invention.

### BRIEF DESCRIPTION OF THE DRAWINGS

FIG. 1 shows the conversion of cyclodextrins to hydroxybutenyl cyclodextrins.

FIG. 2 shows the conversion of hydroxybutenyl cyclodextrins to sulfonated hydroxybutenyl cyclodextrins.

FIG. 3 shows the release of tamoxifen (% maximum) from liquid filled hard gelatin capsules at pH 6.0.

FIG. 4 shows the release of tamoxifen (ppm) from liquid filled hard gelatin capsules at pH 6.0.

FIG. 5 shows the release of tamoxifen (% maximum) from liquid filled hard gelatin capsules at pH 7.4.

FIG. 6 shows the release of tamoxifen (ppm) from liquid filled hard gelatin capsules at pH 7.4.

FIG. 7 shows the oral bioavailability of tamoxifen obtained with Sprague Dawley rats.

FIG. 8 shows the oral bioavailability of tamoxifen for animals dosed with tamoxifen:HBenBCD complexes. Tamoxifen base-tamoxifen citrate treatment groups have been pooled on the basis of time of dosing-dietary status.

FIG. 9 shows the calculated tamoxifen oral bioavailability versus experimental oral bioavailability obtained from a stepwise least squares regression factorial model.

### DETAILED DESCRIPTION OF THE INVENTION

The present invention may be understood more readily by reference to the following detailed description of the invention and the examples provided therein. It is to be understood that this invention is not limited to the specific methods, formulations, and conditions described, as such may vary. It is also to be understood that the terminology used herein is for the purpose of describing particular aspects only and is not intended to be limiting.

In order that the present invention may be more readily understood, certain terms are first defined. Additional definitions are set forth throughout the detailed description.

The singular forms “a,” “an,” and “the” include plural referents unless the context clearly dictates otherwise.

The term “hydroxybutenyl cyclodextrin” refers to all forms of hydroxybutenyl cyclodextrins, including hydroxybutenyl-α, β, or γ-cyclodextrins as well as higher oligomers containing up to about twelve glucose monomers. Columns 5-12 of U.S. Pat. No. 6,479,467 disclose the preparation of hydroxybutenyl cyclodextrins and methods of their use; these sections are hereby incorporated by reference. The term “hydroxybutenyl cyclodextrin” also encompasses derivatives thereof, including sulfonated hydroxybutenyl cyclodextrins such as, for example, sulfonated hydroxybutenyl-α, β, or γ-cyclodextrins.

The term “hydroxybutenyl cyclodextrin derivatives” refers to hydroxybutenyl cyclodextrins that have been further elaborated by attachment of substituents to the hydroxyls of the cyclodextrin ring and/or hydroxybutenyl substituent or by manipulation of the olefin of the hydroxybutenyl substituent. Examples of hydroxybutenyl cyclodextrin derivatives include sulfonated hydroxybutenyl-α, β, or γ-cyclodextrins. Columns 5-13 of U.S. Pat. No. 6,610,671 disclose the preparation of hydroxybutenyl cyclodextrin derivatives and methods of their use; these sections are hereby incorporated by reference.

The term “compound” refers to any chemical entity with poor water solubility, whether natural or synthetic, and from any source or origin. Examples include pharmaceutically active agents, personal care agents, nutraceutical agents or cosmetic agents. A compound can serve as a guest molecule, for example, in an inclusion complex (see below).

The term “complex” or “inclusion complex” refers to a combination of a compound as defined above and a cyclodextrin wherein the compound or a portion thereof is associated with the cyclodextrin. Typically, the compound, or guest molecule, is included within the cavity of the cyclodextrin, or host molecule, wherein the cavity of the cyclodextrin is the space created by the cyclodextrin torus and the cyclodextrin substituents.

The term “mixture” refers to a combination of a compound as defined above and a cyclodextrin mixed in such a manner that the compound is not substantially included within the cyclodextrin cavity. One example of such a mixture occurs when the compound and the cyclodextrin are physically mixed, for example in a mill or blender. Another example of a mixture is when both the compound and the cyclodextrin are dissolved in a common solvent that will compete with the compound for the cyclodextrin cavity space such that the solvent occupies the cyclodextrin cavity and there is little association of the compound with the cyclodextrin. When a compound-cyclodextrin mixture is placed in a aqueous solvent, such as in a physiological environment, a complex can form in situ provided the rate of complex formation is faster than alternate events such as drug precipitation.

The term “metabolites” refers to compounds (e.g., active species) produced upon introduction of the compounds of the invention into a biological system.

The term “analogs” refers to structurally similar compounds that share at least one biological property.

The compositions of the present invention comprise one or more CD derivatives and one or more water-miscible organic solvents. In some embodiments, the water-miscible organic solvents are compatible for use with soft or hard filled capsules. Any water-miscible organic solvent can be used. Examples include, but are not limited to water-miscible synthetic polymers, water-miscible natural polymers, water-miscible semisynthetic polymers, alcohols, polyols, or mixtures of any of the foregoing. Examples of water-miscible organic solvents useful in the present invention include but are not limited to polyethylene glycol, polyethylene glycol-polyethylene glycol copolymers, polyethylene glycol-polyethylene glycol block copolymers, N-methyl-2-pyrrolidone, polyvinylpyrrolidone, polyvinyl alcohol, ethanol, propylene glycol, glycerin, or combination of any of the foregoing. Examples of water-miscible polyethylene glycols (PEG) useful in the present invention include, but are not limited to PEG 200, PEG 300, PEG 400, PEG 600, PEG 1000, PEG 1500, PEG 2000, PEG 3400, PEG 4600, PEG 8000, PEG 10000, or mixtures of any of the foregoing. The PEGs can contain any of a variety of end groups including hydroxyl, methyl, ethyl, butyl, ester, amine, or carboxylate, or combinations thereof. In some embodiments, the polyethylene glycol is PEG 300, PEG 400, PEG 600, PEG 1000 having hydroxyl end groups. All combinations of any of the foregoing polymers, molecular weights, and end groups are within the present invention. In some embodiments, the solvent is a polyethylene glycol optionally containing from about 0 to about 60 wt % of an acceptable cosolvent or cosolvent mixture.

The compounds of the present invention can be any chemical entity with poor water solubility. The compounds can sparingly soluble in water, slightly soluble in water, very slightly soluble in water or insoluble in water. In certain embodiments, the amount of water needed to dissolve one gram of a compound will range from 30 to 100 milliliters (mLs). In some embodiments, the amount of water needed to dissolve one gram of a compound will range from 100 to 1000 mLs. In other embodiments, the amount of water needed to dissolve one gram of a compound will range from 1000 to 10,000 mLs. In additional embodiments, the amount of water needed to dissolve one gram of a compound will be greater than 10,000 mLs.

In certain embodiments, the compositions of the invention have a water content that is sufficiently low such that the compositions can be placed into water-soluble capsules without dissolving the capsules. The water content of the water-miscible organic solvent is frequently the principal source of water in the compositions of the invention. Typically, the solvent or solvent mixture used contains 10% (w/w) or less water. In some embodiments, the solvent or solvent mixture used contains 5 wt % or less water or 2 wt % or less water. In other embodiments, the solvent or solvent mixture used contains from about 0% to about 3 wt % water.

In some embodiments in which the composition contains a pharmaceutically active compound or a salt, structural analog or metabolite thereof, such compounds can in some cases be a source of water added to liquid or semi-solid oral pharmaceutical formulations. For example, a hydrated salt of a pharmaceutical salt introduces water in some embodiments. Similarly, buffers, surfactants, or cyclodextrin derivatives are each sources of added water to compositions in some embodiments. Thus, the composition may contain more water than the solvent or solvent mixture used. Those skilled in the art will recognize that preferred amounts of water and organic solvents in solvent mixtures will depend upon a number of factors such as desirable solution viscosity, the presence or absence of buffers, the nature of the pharmaceutically active compound, the nature of the cyclodextrin derivative, the presence or absence of surfactants, compatibility with physiological fluids, and compatibility/stability with the filled capsule. In some embodiments, the compositions contain 10% wt % or less water; in other embodiments, 5 wt % or less water or 2 wt % or less water. In additional embodiments, the composition contains from about 0% to about 2 wt % water, or from about 0% to about 5 wt % water, or from about 0% to about 10 wt % water.

The compositions also contain a CD derivative. The CD derivative is derived from a CD of any ring size, including but not limited to α, β, or γ-cyclodextrins. The CD derivative chosen for the composition is soluble in the water-miscible organic solvents or solvent mixtures selected. In some embodiments of the present invention, the cyclodextrin derivative is both water soluble and soluble in water-miscible organic solvents or solvent mixtures. In some embodiments, the cyclodextrin derivatives are hydroxyalkyl cyclodextrins, alkyl cyclodextrins, hydroxyalkenyl cyclodextrins, sulfohydroxyalkyl cyclodextrins, or mixtures thereof. Examples of hydroxyalkyl cyclodextrins include hydroxyethyl cyclodextrins, hydroxypropyl cyclodextrins, and hydroxybutyl cyclodextrins. Examples of alkyl cyclodextrins include methyl cyclodextrin or ethyl cyclodextrin. Hydroxybutenyl cyclodextrins are examples of hydroxyalkenyl cyclodextrin. Sulfonated hydroxybutenyl cyclodextrins are examples of sulfohydroxybutyl cyclodextrins. Mixtures of different types of CD derivatives and CDs and CD derivatives are also within the present invention.

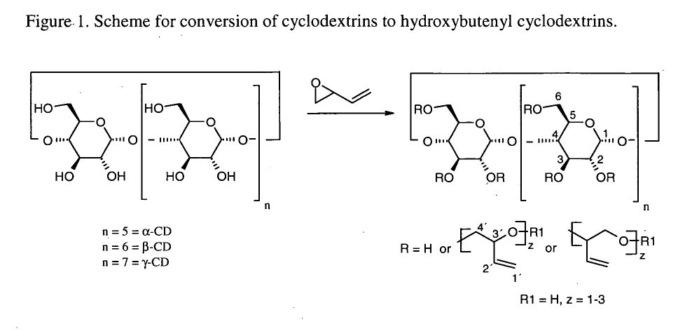

In some embodiments, the cyclodextrin derivatives are hydroxybutenyl cyclodextrins or derivatives thereof. In some embodiments, the hydroxybutenyl cyclodextrins are hydroxybutenyl-α, β, or γ-cyclodextrins (FIG. 1). In some embodiments, the hydroxybutenyl cyclodextrins are hydroxybutenyl-β-cyclodextrins having a molar substitution (MS, wherein MS is the total number of substitutents attached to the CD) from about 1 to about 12. In some embodiments, the hydroxybutenyl cyclodextrins are hydroxybutenyl-β-cyclodextrins with a MS from about 3 to about 10. In some embodiments, the hydroxybutenyl cyclodextrins are hydroxybutenyl-β-cyclodextrins, are water soluble and have a MS from about 4 to about 7. In some embodiments, the hydroxybutenyl cyclodextrins are hydroxybutenyl-β-cyclodextrins that are water soluble and have a MS from about 4.5 to about 5.5.

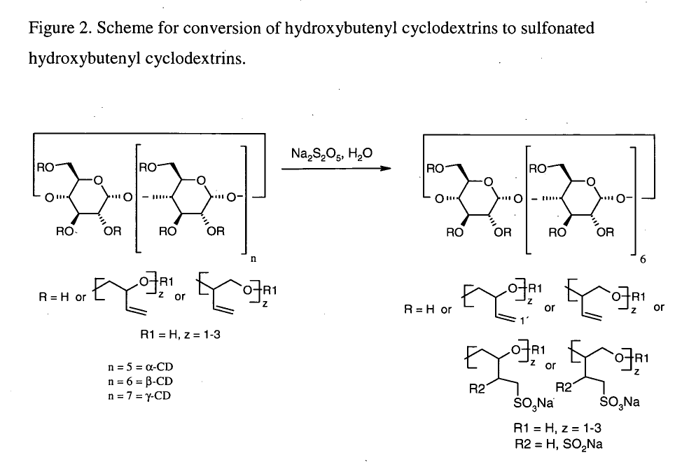

In certain embodiments, the hydroxybutenyl cyclodextrin derivatives are sulfonated hydroxybutenyl-α, β, or γ-cyclodextrins (FIG. 2). In some embodiments, the sulfonated hydroxybutenyl cyclodextrins are sulfonated hydroxybutenyl-β-cyclodextrins comprising at least one hydroxybutyl sulfonate substitutent. In some embodiments, the sulfonated hydroxybutenyl-β-cyclodextrins have a MS of hydroxybutyl sulfonate from about 0.02 to about 7. In other embodiments, the hydroxybutenyl-β-cyclodextrins have a MS of hydroxybutyl sulfonate from about 0.05 to about 5 or from about 0.1 to about 2. In the case of sulfonated hydroxybutenyl-α, β, or γ-cyclodextrins, those skilled in the art will recognize that these cyclodextrin ethers contain both hydroxybutenyl substitutents and hydroxybutyl sulfonate substitutents. In this case, the total MS is provided by the sum of the hydroxybutenyl MS and the hydroxybutyl sulfonate. In some embodiments, the MS is from about 0.02 to about 12. Cyclodextrin ethers containing at least one hydroxybutyl sulfonate substitutent can also further comprise additional alkyl, sulfinate, or disulfonate substitutents.

In some embodiments of the present invention, the composition also contains one or more additional compound or molecule that can form an inclusion complex with the CD derivative. The additional compound or molecule can be present in a state of complexation from the CD derivative, as a physical mixture free from complexation, or present in both states. The additional compounds may be one or more of any compound that can form inclusion complexes with the CD.

In some embodiments, the CD derivative serves to solubilize and stabilize the compound when the composition is added to an aqueous environment as well as provide for enhanced and/or sustained release and to increase bioavailability in the appropriate physiological environment. In certain embodiments, the compositions of the present invention can provide a higher concentration of compound per unit volume than conventional formulations when introduced into an aqueous environment.

In certain embodiments, a compound can form an inclusion complex with the CD derivative when the composition of the invention is placed in an aqueous environment. For example, a pharmaceutical compound existing as a mixture with a CD derivative in a water-miscible organic solvent can form an inclusion complex with the CD derivative when introduced into an aqueous physiological environment such as the stomach. In certain embodiments, the compound forms a complex with the CD derivative when the composition is introduced into an aqueous environment at a faster rate than the compound precipitates, rendering the compound more bioavailable.

In certain embodiments, the additional compound or molecule can be a pharmaceutically active compound. Throughout this application the terms “drug,” “pharmaceutical,” and “pharmaceutically active compound” each refer to any substance which, when administered to a human or animal under conditions effective to cause absorption to the bloodstream, causes a therapeutic or prophylactic effect.

The pharmaceutically active compounds of the present invention can be therapeutically active agents or pharmaceutically acceptable salts or metabolites thereof. Any pharmaceutically active compound is within the present invention. In some embodiments, the pharmaceutically active agents are antineoplastics, antiviral agents, antidiabetic agents, antidepressants, antifungal agents, antibacterial agents, antimigrane, antiprotozoal agents, antisense agents, androgens, estrogens, sedatives, serotonin antagonists, narcotic antagonists, narcotic agonists, proteins, peptides, steroids, tranquilizers, antipsychotics, antidepressants, antiallergics, antianginals, antiarthritics, antiasthmatics, antidiabetics, antidiarrheal drugs, anticonvulsants, antigout drugs, antihistamines, antipruritics, anticoagulants, emetics, antiemetics, antispasmondics, appetite suppressants, neuroactive substances, neurotransmitter agonists, antagonists, receptor blockers and reuptake modulators, beta-adrenergic blockers, calcium channel blockers, disulfarim and disulfarim-like drugs, muscle relaxants, analgesics, antipyretics, stimulants, anticholinesterase agents, parasympathomimetic agents, hormones, anticoagulants, antithrombotics, thrombolytics, immunoglobulins, immunosuppressants, hormone agonists/antagonists, vitamins, antimicrobial agents, antineoplastics, antacids, digestants, laxatives, cathartics, antiseptics, diuretics, disinfectants, fungicides, ectoparasiticides, antiparasitics, heavy metals, heavy metal antagonists, chelating agents, gases and vapors, alkaloids, salts, ions, autacoids, digitalis, cardiac glycosides, antiarrhythmics, antihypertensives, vasodilators, vasoconstrictors, antimuscarinics, ganglionic stimulating agents, ganglionic blocking agents, neuromuscular blocking agents, adrenergic nerve inhibitors, anti-oxidants, vitamins, cosmetics, anti-inflammatories, wound care products, antithrombogenic agents, antitumoral agents, antithrombogenic agents, antiangiogenic agents, anesthetics, antigenic agents, wound healing agents, plant extracts, growth factors, emollients, humectants, rejection/anti-rejection drugs, spermicides, conditioners, antibacterial agents, antifungal agents, antiviral agents, antibiotics, tranquilizers, cholesterol-reducing drugs, agents for treatment of Parkinson's or Alzheimer's disease, vitamins/nutritional factors, antitussives, histamine-blocking drugs, monoamine oxidase inhibitor, or pharmaceutically acceptable salts or metabolites of any of the foregoing. In some embodiments, the pharmaceutically active drugs are hydrophobic, poorly water-soluble drugs.

Non-limiting examples of pharmaceutically active compounds useful in embodiments of the present invention include: abacavir, acarbose, acebutolol, acetazolamide, acetohexamide, acrivastine, acutretin, acyclovir, alatrofloxacin, albendazole, albuterol, alclofenac, alendronate, allopurinol, aloxiprin, alprazolam, alprenolol, alprostadil, amantadine, amiloride, aminoglutethimide, amiodarone, amitriptyline, amlodipine, amodiaquine, amoxapine, amoxapine, amphetamine, amphotericin, amprenavir, amrinone, amsacrine, amyl nitrate, amylobarbital, amylobarbitone, anastrozole, arzoxifene, aspirin, astemizole, atenolol, atorvastatin, atovaquone, atropine, auranofin, azapropazone, azathioprine, azelastine, azithromycin, baclofen, barbital, barbitone, becaplermin, beclamide, beclomethasone, bendrofluazide, benethamine, benethamine penicillin, benezepril, benidipine, benorylate, bentazepam, benzhexol, benznidazole, benzonatate, benztropine, bephenium hydroxynaphthoate, betamethasone, bexarotene, bezafibrate, bicalutamide, bifonazole, biperiden, bisacodyl, bisanthrene, bovine growth hormone, bromazepam, bromfenac, bromocriptine, bromocriptine mesylate, bromperidol, brompheniramine, brotizolam, budesonide, bumetanide, bupropion, busulphan, butenafine, butobarbital, butobarbitone, butoconazole, butoconazole nitrate, calcifediol, calciprotiene, calcitonin, calcitriol, cambendazole, camidazole, camptothecan, camptothecin, candesartan, capecitabine, capsacin, capsaicin, captopril, carbamazepine, carbimazole, carbinoxamine, carbromal, carotenes, cefazolin, cefoxitin sodium, celecoxib, cephadrine, cephalexin, cerivistatin, cetrizine, chlopheniramine, chlophenisamine, chloproguanil, chlorambucil, chlordiazepoxide, chlormethiazole, chloroquine, chlorothiazide, chlorpromazine, chlorpropamide, chlorprothiocene, chlorprothixene, chlorthalidone, cholecalciferol, cilostazol, cimetidine, cinnarizine, cinoxacin, ciprofloxacin, cisapride, citalopram, citrizine, clarithromycin, clemastine, clemastine fumarate, clemizole, clenbuterol, clinofibrate, clioquinol, clobazam, clofazimine, clofibrate, clomiphene, clomipramine, clonazepam, clopidrogel, clotiazepam, clotrimazole, cloxacillin, clozapine, codeine, conjugated estrogens, cortisone acetate, cortisone acetate, cromalyn sodium, cromoglicate, cromolyn, cyclizine, cyclosporin, cyproheptadine, dacarbazine, danazol, dantrolene, darodipine, decoquinate, delavirdine, demeclocycline, desoxymethasone, dexamphetamine, dexanabinol, dexchlopheniramine, dexfenfluramine, dextropropyoxyphene, diamorphine, diazepam, diazoxide, dichlorophen, diclofenac, dicloxacillin, dicoumarol, dicumarol, didanosine, diethylpropion, diflunisal, digitoxin, digoxin, dihydro epiandrosterone, dihydrocodeine, dihydroergotamine, dihydroergotamine mesylate, dihydrotachysterol, diiodohydroxyquinoline, dilitazem, diloxanidefuroate, dimenhydrinate, dinitolmide, diphenhydramine, diphenooxylate, diphenylimidazole, diphenylpyrallin, dipyridamole, dirithromycin, disopyramide, divalproen, docetaxel, doconazole, docusate, dolasetron, domperidone, donepezil, doxercalciferol, doxazosin, doxycycline, doxorubicin, droloxifene, dronabinol, droperidol, dutasteride, econazole, econazole nitrate, editronate, efavirenz, elanapril, ellipticine, enalapril, enkephalin, enoxacin, enoximone, enrofloxacin, epalrestate, eperisone, ephedrine, eposartan, eposartan losartan, ergocalciferol, ergotamine, erythromycin, erythropoietin, essential fatty acids, estramustine, ethacrynic acid, ethambutol, ethinamate, ethinyloestradiol, ethionamide, ethopropazine, ethotoin, etodolac, etoperidone, etoposide, etretinate, exemestane, fadrozole, famcyclovir, famotidine, felbamate, felodipine, fenbendazole, fenbufen, fenfluramine, fenofibrate, fenolclopam, fenoldopam, fenoprofen, fenoprofen calcium, fentanyl, fenticonazole, fexofenadine, finasteride, flecainide, fluconazole, flucortolone, flucytosine, fludrocortisone, flunanisone, flunarizine, flunisolide, flunitrazepam, fluopromazine, fluoxetine, fluoxymisterone, flupenthixol decanoate, flupentixol, flupentixol decanoate, fluphenazine, fluphenazine decanoate, flurazepam, flurbiprofen, flurithromycin, fluticasone, fluvastatin, formestane, foscarnet, fosinopril, fosphenytoin, frovatriptan, frusemide, fumagillin, furazolidone, furosemide, furzolidone, gabapentin, gancyclovir, gemfibrozil, gentamycin, glibenclamide, gliclazide, glipizide, glucagon, glybenclamide, glyburide, glyceryl trinitrate, glymepiride, glymepride, granisetron, granulocyte stimulating factor, grepafloxacin, griseofulvin, goserelin, guanabenz, halofantrine, haloperidol, hydrocortisone, hyoscyamine, ibufenac, ibuprofen, imipenem, idarubicin, indinavir, indivir, indomethacin, insulin, interferon, pegylated interferon, interleukin-3, irbesartan, irinotecan, isoconazole, isosorbide dinitrate, isosorbide mononitrate, isotretinoin, isoxazole, isradipine, itraconazole, ivermectin, ketoconazole, ketoprofen, ketorolac, ketotifen, labetalol, lamivudine, lamotrigine, lanatoside C, lanosprazole, leflunomide, letrozole, levofloxacin, levothyroxine, lisinopril, linezolide, lombazole, lomefloxacin, lomustine, loperamide, lopinavir, loratadine, lorazepam, lorefloxacin, lormetazepam, losartan, lotrimin, lovastatin, L-thryroxine, lysuride, lysuride maleate, maprotiline, mazindol, mebendazole, meclofenamic acid, meclozine, medazepam, medigoxin, medroxyprogesterone acetate, mefenamic acid, mefloquine, megesterol acetate, melonicam, meloxicam, melphalan, mepacrine, mepenzolate bromide, meprobamate, meptazinol, mercaptopurine, mesalazine, mesoridazine, mesoridiazine, mestranol, metformin, methadone, methaqualone, methoin, methotrexate, methoxsalen, methsuximide, methylphenidate, methylphenobarbital, methylphenobarbitone, methylprednisolone, methyltestosterone, methysergide, methysergide maleate, metoclopramide, metolazone, metoprolol, metronidazole, mianserin, miconazole, midazolam, miglitol, minoxidil, mitomycin, mitotane, mitoxantrone, mofetil, molindone, montelukast, morphine, mortriptyline, moxifloxacin, mycophenolate, nabumetone, nadolol, nalbuphine, nalidixic acid, naproxen, naratriptan, natamycin, nedocromil sodium, nefazodone, nelfinavir, nerteporfin, neutontin, nevirapine, nicardipine, nicotine, nicoumalone, nifedipine, nilutamide, nimesulide, nimodipine, nimorazole, nisoldipine, nitrazepam, nitrofurantoin, nitrofurazone, nizatidine, non-essential fatty acids, norethisterone, norfloxacin, norgestrel, nortriptyline HCl, nystatin, oestradiol, ofloxacin, olanzapine, omeprazole, ondansetron, oprelvekin, ornidazole, orconazole, ospemifene, oxacillin, oxamniquine, oxantel, oxantel embonate, oxaprozin, oxatomide, oxazepam, oxcarbazepine, oxfendazole, oxiconazole, oxmetidine, oxprenolol, oxybutynin, oxyphenbutazone, oxyphencylcimine, paclitaxel, pamidronate, paramethadione, parconazole, paricalcitol, paroxetine, penicillins, pentaerythritol tetranitrate, pertazocine, pentobarbital, pentobarbitone, pentoxifylline, perchloperazine, perfloxacin, pericyclovir, perphenazine, perphenazine pimozide, phenacemide, phenbenzanine, phenindione, pheniramine, phenobarbital, phenobarbitone, phenoxybenzamine, phensuximide, phentermine, phenylalanine, phenylbutazone, phenytoin, physostigmine, phytonodione, pimozide, pindolol, pioglitazone, piroxicam, pizotifen, pizotifen maleate, posaconazole, pramipexol, pramipexole, pranlukast, pravastatin, praziquantel, prazosin, prednisolone, prednisone, pregabalin, primidone, probenecid, probucol, procarbazine, prochlorperazine, progesterone, proguanil, propofol, propranolol, propylthiouracil, pseudoephedrine, pyrantel, pyrantel embonate, pyridostigmine, pyrimethamine, quetiapine, quinapril, quinidine, quinine, rabeprazole, raloxifene, ranitidine, ravuconazole, recombinant human growth hormone, refocoxib, remifentanil, repaglinide, reserpine, residronate, retinoids, ricobendazole, rifabutin, rifabutine, rifampicin, rifampin, rifapentine, rimantadine, rimexolone, risperodone, ritonavir, rizatriptan, rizatriptan benzoate, ropinirole, rosiglitazone, roxatidine, roxithromycin, salbutamol, salmon calcitonin (sCT), saquinavir, selegiline, sertindole, sertraline, sibutramine, sildenafil, simvastatin, sirolimus, sodium cefazoline, somatostatin, sparfloxacin, spiramycins, spironolactone, stanozolol, stavudine, stavueline, stiboestrol, sulconazole, sulfabenzamide, sulfacetamide, sulfadiazine, sulfadoxine, sulfafurazole, sulfarnerazine, sulfamethoxazole, sulfapyridine, sulfasalazine, sulindac, sulphabenzamide, sulphacetamide, sulphadiazine, sulphadoxine, sulphafurazole, sulphamerazine, sulphamethoxazole, sulphapyridine, sulphasalazine, sulphin-pyrazone, sulpiride, sulthiame, sumatriptan, tacrine, tacrolimus, tamoxifen, tamsulosin, targretin, tazarotene, telmisartan, temazepam, teniposide, terazosin, terbinafine HCl, terbutaline, terbutaline sulfate, terconazole, terenadine, terfenadine, testolactone, testosterone, tetracycline, tetrahydrocannabinol, tetramisole, thiabendazole, thioguanine, thioridazine, tiagabine, tibolone, ticlidopine, ticlopidine, tiludronate, timolol, tinidazole, tioconazole, tirofibran, tizanidine, tolazamide, tolbutamide, tolcapone, tolmetin, tolterodine, topiramate, topotecan, toremifene, tramadol, trazodone, tretinoin, triamcinolone, triamterene, triazolam, trifluoperazine, trimethoprim, trimipramine, troglitazone, tromethamine, tropicamide, trovafloxacin, tubulazole, tumor necrosisi factor, undecenoic acid, ursodeoxycholic acid, valacylcovir, valconazole, valproic acid, valsartan, vancomycin, vasopressin, venlafaxine HCl, verteporfin, vigabatrin, vinblastine, vincristine, vinorelbine, vitamin A, vitamin B2, vitamin D, vitamin E and vitamin K, vitamin K5, vitamin K6, vitamin K7, vitamin K-S (II), voriconazole, zafirlukast, zileuton, ziprasidone, zolmitriptan, zolpidem, and zopiclone.

In some embodiments, the pharmaceutically active compound can be one or more selective estrogen modulators (e.g. arzoxifene, droloxifene, ospemifene, raloxifene, tamoxifen, toremifene), aromatase inhibitors (e.g. anastrozole, formestane, exemestane, letrozole), antineoplastics (e.g. camptothecin, docetaxel, doxorubicin, etoposide, paclitaxel, tretinoin), antiviral agents (e.g. amprenavir, lopinavir, nelfinavir, ritonavir, saquinavir), antifungal agents (e.g. itraconazole, ketoconazole, posaconazole, ravuconazole, voriconazole), and immunosuppressants (e.g. cyclosporin, sirolimus).

In some embodiments, a pharmaceutically active compound can be a pharmaceutically acceptable salt. Example include but are not limited to non-toxic salts, such as salts from organic acids (e.g., formic, acetic, propionic, trifluoroacetic, citric, maleic, tartaric, ascorbic, methanesulfonic, benzenesulfonic, toluenesulfonic acids), from inorganic acids (e.g., hydrochloric, hydrobromic, sulfuric, or phosphoric acids), and amino acids (e.g., aspartic or glutamic acids). In some embodiments, the pharmaceutically acceptable salt is a citrate, tartrate, acetate, propionate, mesylate, or HCl salt. The pharmaceutically acceptable salts of the drugs of the present invention can be prepared by any effective means. In some embodiments, a solution or a suspension of a free base of one or more pharmaceutically active compound is treated with about one equivalent or slight excess of the pharmaceutically acceptable acid. The resulting salt is then isolated by conventional methods.

An amount of one or more pharmaceutically active compound or pharmaceutically acceptable salts or metabolites thereof can be used such that the formulations of the present invention provide the desired therapeutic effect. In some embodiments, they are administered one to four times a day with a unit dosage of 0.25 to 2000 milligrams (mg) in human patients. This dosage can be properly varied depending on the specific drug and age, body weight, and medical condition of the patient. In some embodiments, one dose of 1-100 mg one time a day is used. In some embodiments, the unit dose is a liquid or semi-solid filled gelatin capsule.

Many of the metabolites of pharmaceutically active compounds and their pharmaceutically acceptable salts are biologically active. For example, tamoxifen and toremifene undergo phase I metabolism in the liver by microsomal cytochrome P450 enzymes. The major metabolites of tamoxifen are N-desmethyltamoxifen and 4-hydroxytamoxifen. The major metabolites of toremifene are N-desmethyltoremifene and deaminohydroxytoremifene (ospemifene). Both 4-hydroxytamoxifen and deaminohydroxytoremifene are biologically active. Pharmaceutically active compounds also include metabolites of drugs or their pharmaceutically acceptable salts.

The compositions of the present invention optionally include additional components. In some embodiments, additional components are useful in achieving or enhancing desired properties of the compositions. One example is one or more surfactants. In some embodiments, the surfactants are water soluble or dispersible non-ionic surfactants. Non-limiting examples of surfactants useful in some embodiments of the present invention include cremophors (eg. Cremophor EL, Cremophor 40), polysorbate 20 (Tween 20), polysorbate 80 (Tween 80), tocopherol polyethylene glycol 1000 succinate (TPGS), Solutol HS-15, sorbitan monooleate (Span 80), alkylpolyglycosides (cf. U.S. Pat. No. 6,077,945), and carbohydrate esters (eg. sucrose acetate isobutyrate, (cf. US 2005/228084) In some embodiments, the surfactant is Tween 20, Tween 80, TPGS, sucrose acetate isobutyrate, a alkylpolyglycoside or a combination of any of the foregoing.

In some embodiments, surfactants serve to modify the stability and release rates of compounds, such as drugs, in a physiological environment. In some embodiments, the surfactant also acts as a P-glycoprotein inhibitor. In some embodiments, the concentration of surfactant in the pharmaceutical formulations of the present invention is from about −15× to +3× of the critical micelle concentration of the surfactant. In some embodiments, the concentration of surfactant is from about −10× to +1× of the critical micelle concentration of the surfactant.

Another example of optional additional compounds is a P-glycoprotein (p-gp) inhibitor. In some embodiments, a water-miscible organic solvent, a cyclodextrin derivative, one or more pharmaceutically active compounds, a surfactant, or any of combination of the foregoing also includes a p-gp inhibitor. In some embodiments, methyl cyclodextrin is a p-gp inhibitor. In some embodiments, polyethylene glycol-polyethylene glycol block copolymers (eg. Pluronics available from BASF), cremophors, TPGS, Solutol, and Polysorbate 80 are p-gp inhibitors. In some embodiments, the pharmaceutically active compound include amiodarone, aldosterone, lidocaine, clomiphene, cefoperazone, cortisol, ceftriaxone, dexamethasone, erythromycin, prednisone, itraconazole, progesterone, chloroquine, tamoxifen, emetine, desipramine, quinidine, trazodone, hydroxychloroquine, dipyridamole, quinacrine, reserpine, quinine, cyclosporin A, bepridil, colchicine, diltiazem, felodipine, quercetin, nifedipine, nitrendipine, terfenadine, tiapamil, verapamil, vitamin A, actinomycin D, etoposide, daunorubicin, mitomycin C, ketoconazole, taxol, vinblastine, devapamil, vincristine, gallopamil, indinavir, emopamil, nelfinavir, emopamil, saquinavir, ritonavir, or bupivacaine, each of which exhibit p-gp inhibitor activity in addition to their therapeutic use. In some embodiments, the pharmaceutically active compound having p-gp inhibitor activity include nelfinavir, quinidine, saquinavir, ritonavir, clomiphene, cyclosporin, indinavir, verapamil, cefoperazone, and ceftriaxone. In some embodiments, a p-gp inhibitor is used which is a pluronic, TPGS, ritonavir, or cyclosporin.

Other examples of optional components in the compositions of the present invention include but are not limited to useful excipients such as organic acids, organic bases, buffers, antioxidants, preservatives, and tonicity adjusters. In some embodiments, the organic acid is acetic, citric, or propionic acid. In some embodiments, the organic base is ethylene diamine, triethanolamine, tris(hydroxymethyl)aminomethane, or butyl amine, In some embodiments, the organic acid is phosphates, acetates, citrates, benzoates, succinates, bicarbonates, and glycine. Examples of antioxidants include but are not limited to ascorbic acid, sodium bisulfite, sodium metabisulfite, monothioglycerol, thiourea, butylated hydroxytoluene, butylated hydroxy anisole, and ethylenediaminetetraacetic acid salts. Preservatives useful in liquid formulations include but are not limited to benzoic acid and its salts, sorbic acid and its salts, alkyl esters of parahydroxybenzoic acid, phenol, chlorobutanol, benzyl alcohol, thimerosal, benzalkonium chloride and cetylpyridinium chloride.

One aspect of this invention is directed to compositions of the present invention that are pharmaceutical formulations suitable for liquid administration. One aspect of this invention is directed to liquid or semi-solid oral pharmaceutical formulations wherein the pharmaceutical formulations are suitable for filling soft or hard shell gelatin capsules. Formulations suitable for filling water-soluble capsules have a water content that is sufficiently low to prevent the formulation from dissolving the capsule.

Liquid formulations include but are not limited to oral solutions, oral suspensions, parenteral solutions, and solutions for subcutaneous administration wherein the water content is less than about 10 wt % or less.

Some embodiments of the present invention are very stable solutions that can be stored under appropriate conditions (from about 5° C. to about room temperature) for periods up to 2 years or longer. In some embodiments in which parenteral administration is the chosen route of administration, intramuscular injection is used.

The present invention also includes methods of making the compositions of the present invention. In some embodiments of preparing the formulations of the present invention, a physical mixture of the cyclodextrin derivative and compound or drug is dissolved directly in the water-miscible organic solvent. In other embodiments, the cyclodextrin derivative is first dissolved in the water-miscible organic solvent followed by the addition of the compound or drug. In yet other embodiments, the compound or drug is first dissolved in the water-miscible organic solvent followed by the addition of the cyclodextrin derivative. Those of skill in the art will know that the order in which the components are added will vary depending upon the nature of the compound or drug, cyclodextrin derivative and water-miscible organic solvent.

In some embodiments, a solid inclusion complex is preformed and then dissolved in the water-miscible organic solvent. In other embodiments, the inclusion complex can be formed after all the components have been added. Physical mixtures can be formed by methods known to those skilled in the art. In some embodiments, the methods provide an intimate physical mixture in which the particle size of the components are reduced. For example, methods such as dry milling can be utilized in the present invention. Dry solid inclusion complexes can also be formed by conventional methods. In some embodiments, the excess amount of drug is added to an aqueous solution of the cyclodextrin derivative and mixed for a period of time sufficient to obtain equilibrium solubility. Aqueous solutions in some embodiments are those in which the water content is at least 20 wt %. Excess drug can be removed and the inclusion complex can be isolated, for example by drying techniques such as spray drying or freeze drying. In some embodiments, the inclusion complex is isolated by precipitation in a solvent in which the complex has minimal solubility. The molar ratio of the inclusion complex components can vary depending upon the initial solution concentration of each component. In some embodiments, the amount of cyclodextrin derivative is such that the molar ratio of drug to cyclodextrin derivative is from about 1:0.1 to about 1:30. In some embodiments, the amount is from about 1:0.5 to about 1:10. In some embodiments, the amount is from about 1:1 to about 1:4.

Liquid formulations of the present invention can be formed by any methods, including conventional methods. For example, the liquid formulation can be formed by adding one or more drugs and one or more cyclodextrin derivatives, in an amount less than or equal to the amount corresponding to equilibrium solubility, directly to the water-miscible organic solvent. In some embodiments, a preformed inclusion complex is added to the water-miscible organic solvent.

The present invention further includes capsules containing the compositions of the present invention. Any effective type of capsule can be used. Any material useful in the preparation of capsule shell can be used in a capsule according to the invention. Nonlimiting examples of materials suitable for the preparation of the capsule shell include soft gelatin, hard gelatin, hydroxypropyl methylcellulose, starch, animal gelatin, agar, fish (piscine) gelatin or a combination thereof. Other suitable materials include: polyvinyl alcohol/polyvinyl acetate copolymers (U.S. Pat. No. 3,300,546); a blend of hydroxybutyl methylcellulose and hydroxypropyl methylcellulose (U.S. Pat. No. 4,765,916); polyvinyl acetate (U.S. Pat. No. 2,560,649, U.S. Pat. No. 3,346,502); water-soluble gelatin (U.S. Pat. No. 3,525,426); polyvinyl alcohol (U.S. Pat. Nos. 3,528,921; 3,534,851; 3,556,765; 3,634,260; 3,671,439; 3,706,670; 3,857,195; 3,877,928; 4,367,156; 4,747,976; 5,270,054); polymers derived from such monomers as vinyl chloride, vinyl alcohol, vinyl pyrrolidone, furan, acrylonitrile, vinyl acetate, methyl acrylate, methyl methacrylate, styrene, vinyl ethyl ether, vinyl propyl ether, acrylamide, ethylene, propylene, acrylic acid, methacrylic acid, maleic anhydride, salts of any of the aforementioned acids and mixtures thereof; glucomannan and optionally another natural polysaccharides with a polyhydric alcohol; polylactide/polyglycolide; arabinoxylan (U.S. Pat. No. 6,388,069); or a combination thereof. Suitable starch capsules can be made and used according to Vilivalam et al. (Pharmaceutical Science & Technology Today 2000, 3, 64-69). A chitosan capsule for colonic delivery can be made and used according Tozaki et al. (Drug Delivery Systems 1997, 12, 311-320). In some embodiments, the capsules include additional components. In some embodiments, for example, the capsule is coated with an enteric coating to promote dissolution of the capsule at a desired pH.

In some embodiments involving liquid filled capsules, the compositions contain less than about 20% suspended solids. In some embodiments, the compositions contain less than about 10% suspended solids. In some embodiments, the composition does not contain solids visible to the eye. The liquid composition in some embodiments has a solution viscosity that will permit filling of capsules. The liquid composition in some embodiments provides for rapid or sustained release of the pharmaceutical active in the desired environment.

In some embodiments involving semi-solid filled capsules, the viscosity of the formulations decreases with increasing temperature. For example, in some embodiments, upon heating from about 40 to about 100° C., the viscosity of the formulation allows filling of capsules. Upon cooling to ambient temperature, the pharmaceutical formulation can have a high solution viscosity or can be in a semi-solid state.

The present invention also includes methods of increasing the bioavailability of a compound comprising formulating the compound with a cyclodextrin derivative and a water-miscible organic solvent, wherein the formulation contains 10 wt % or less water. In some embodiments, the formulation is administered to a subject. Examples of suitable subjects include, but are not limited to, invertebrates, vertebrates, and mammals. In one embodiment, the subject is a human. In some embodiments, the compound is a pharmaceutically active compound.

Any method of administration can be used with the compositions of the present invention. Examples of such methods include, but are not limited to, oral administration (e.g. buccal or sublingual administration), ingestion through intestinal absorption, anal administration, rectal administration, administration as a suppository, topical application, aerosol application, inhalation, intraperitoneal administration, intravenous administration, transdermal administration, intradermal administration, subdermal administration, intramuscular administration, intrauterine administration, vaginal administration, administration into a body cavity, surgical administration at the location of a tumor or internal injury, administration into the lumen or parenchyma of an organ, and parenteral administration. Any technique can be used in the method of administration. Examples of techniques useful in the various forms of administrations above include, but are not limited to, topical application, ingestion, surgical administration, injections, sprays, transdermal delivery devices, osmotic pumps, depositing directly on a desired site, or other means familiar to one of ordinary skill in the art. Sites of application can be external, such as on the epidermis, or internal, for example a gastric ulcer, a surgical field, or elsewhere.

The compositions of the present invention can be applied in any form. Examples include, but are not limited to, creams, gels, solutions, suspensions, liposomes, particles, or other means known to one of skill in the art of formulation and delivery of therapeutic and cosmetic compounds. Some examples of appropriate formulations for subcutaneous administration include but are not limited to implants, depot, needles, capsules, and osmotic pumps. Some examples of appropriate formulations for vaginal administration include but are not limited to creams and rings. Some examples of appropriate formulations for oral administration include but are not limited to: pills, liquids, syrups, and suspensions. Some examples of appropriate formulations for transdermal administration include but are not limited to gels, creams, pastes, patches, sprays, and gels. Some examples of appropriate delivery mechanisms for subcutaneous administration include but are not limited to implants, depots, needles, capsules, and osmotic pumps. Formulations suitable for parenteral administration include but are not limited to aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. Extemporaneous injection solutions and suspensions may be prepared, for example, from sterile powders, granules and tablets.

The invention has been described in detail with particular reference to preferred embodiments thereof, but it will be understood that variations and modifications can be effected within the spirit and scope of the invention. In the drawings and specification, there have been disclosed typical preferred embodiments of the invention. Although specific terms are employed, they are used in a generic and descriptive sense only and not for purposes of limitation, the scope of the invention being set forth in the following claims.

### EXAMPLES

The following examples are offered for illustrative purposes only.

Hydroxybutenyl cyclodextrins (HBenCD) were prepared according to the general methods described in U.S. Pat. No. 6,479,467. Sulfonated hydroxybutenyl cyclodextrins (SulfoHBenCD) were prepared according to the general methods described in U.S. Pat. No. 6,610,671. All of the cyclodextrin derivatives were dried at 10-15 mm Hg at room temperature for 14 to 60 h prior to use. All of the drugs were obtained from Apin Chemicals and characterized prior to use.

### Example 1

### Solubility of Hydroxybutenyl Cyclodextrins in Selected Water-Miscible Organic Solvents

In the following experiments, PEG 400 and propylene glycol were dried over molecular sieves prior to use and the H₂O content was determined by Karl Fisher titration (PEG 400=0.238 wt % H₂O; PG=0.179 wt % H₂O). The ethanol (EtOH) was absolute EtOH and the water was prefiltered through a Millipore Milli-Q Water System. The hydroxybutenyl-β-cyclodextrin (MS=4.7, HBenβCD_{4.7}) was carefully dried prior to use.

The appropriate solvent and HBenβCD_{4.7} were weighed into glass vials with screw caps. The samples were briefly (ca. 2 min) heated to 60° C. and then allowed to mix by gently rolling the vials overnight. The solubility of the HBenβCD_{4.7} in the selected solvent was determined after the initial heating and after rolling overnight. Table 1 provides the composition of the mixtures evaluated for solubility.

TABLE 1
Composition of hydroxybutenyl-β-cyclodextrin and water-miscible
organic solvents or solvent mixtures evaluated for solubility

| | wt % | wt % PEG | wt % | wt % | wt % |
|---|---|---|---|---|---|
| Sample | HBenβCD_{4.7} | 400 | PG | EtoH | H₂O |
| 1 | 5 | 95 | | | |
| 2 | 5 | | 95 | | |
| 3 | 10 | 90 | | | |
| 4 | 10 | | 90 | | |
| 5 | 20 | 80 | | | |
| 6 | 18 | | 82 | | |
| 7 | 30 | 70 | | | |
| 8 | 30 | | 70 | | |
| 9 | 40 | 60 | | | |
| 10 | 40 | | 60 | | |
| 11 | 20 | 76 | | | 4 |
| 12 | 20 | | 75 | 5 | |
| 13 | 40 | 55 | | | 5 |
| 14 | 40 | 57 | | 3 | |

In very case, the HBenβCD_{4.7} was determined to be dissolved by visual inspection (clear solution, no visible particles) after mixing overnight. In the case of samples 1-8, dissolution was observed by visual inspection after heating at 60° C. for ca. 2 minutes. After storage of the solutions at ambient temperature for 5 weeks, all samples still were observed by visual inspection to be completely dissolved.

The results illustrate that hydroxybutenyl-β-cyclodextrins are completely soluble in PEG 400 and propylene glycol over a broad concentration of hydroxybutenyl-β-cyclodextrin (Samples 1-10). Additionally, inclusion of EtOH or water into the system had no adverse effects on the solubility of hydroxybutenyl-β-cyclodextrin.

### Example 2

### Initial Screening for Solubility of Selected Drugs in Hydroxybutenyl Cyclodextrins:Water-Miscible Organic Formulations

In the following experiments, the water-miscible solvents were dried as described in example 1. The hydroxybutenyl-β-cyclodextrins (HBenβCD_{6.6} and HBenβCD_{5.0}) and the sulfonated hydroxybutenyl-β-cyclodextrins (MS_{sulfonate}=0.3, MS_{butentyl}=4.4) were carefully dried prior to use. The following stock solutions were prepared:

1. HBenβCD_{5.0} (30 wt %) and PEG 400 (70 wt %)

2. HBenβCD_{5.0} (30 wt %), PEG 400 (66.2 wt %), and EtOH (3.8 wt %)

3. HBenβCD_{5.0} (30 wt %), PEG 400 (66.2 wt %), and PG (3.8 wt %)

4. HBenβCD_{6.6} (32 wt %) and PEG 400 (68 wt %)

5. HBenβCD_{6.6} (32 wt %), PEG 400 (64.6 wt %), and EtOH (3.4 770629 wt %)

6. HBenβCD_{6.6} (32 wt %), PEG 400 (64.6 wt %), and PG (3.4 wt %)

7. SulfoHBenCD (29 wt %), PEG 400 (67.6 wt %), and EtOH (3.4 wt %)

Various drugs (20-22 mg) were weighed into different glass vials with screw caps. To each vial was added 1 gram (g) ±0.02 g of stock solution. The vial contents were mixed for ca. 48 h. Vials were inspected visually to determine whether the drugs were dissolved. For any vials containing drugs that were not completely dissolved additional stock solution and/or water were added until visual inspection showed dissolution. Mixing and solvent adjustments were continued until the drug was completely dissolved. The solvent concentrations required to solubilize the drugs are summarized in Table 2.

TABLE 2
Initial solubility screening of selected drugs in cyclodextrin
derivatives:water-miscible organic solvents

| | Stock | Stock | Stock | Stock | Stock | Stock | Stock |
|---|---|---|---|---|---|---|---|
| | #1 | #2 | #3 | #4 | #5 | #6 | #7 |
| Danazol | 1.1 | 1.1 | 1.1 | 1.1 | 1.1 | 1.1 | 1.2 |
| Exemestane | 1.5 | 1.5 | 1.5 | 1.5 | 1.5 | 1.5 | 1.5 |
| Paclitaxel | 1.0 | 1.0 | 1.0 | 1.0 | 1.0 | 1.0 | 1.0 |
| Ritonavir | 1.2 | 1.4 | 1.3 | 1.4 | 1.4 | 1.4 | 1.1 |
| Anastrozole | 1.0 | 1.0 | 1.0 | 1.0 | 1.0 | 1.0 | 1.0 |
| ¹Raloxifene | 1.5 | 1.4 | 1.6 | 1.6 | 1.6 | 1.6 | 1.1 |
| HCL | | | | | | | |
| ²Tamoxifene | 1.5 | 1.3 | 1.3 | 1.5 | 1.3 | 1.3 | 1.3 |
| Citrate | | | | | | | |

The values reported are grams of stock solution required to solubilize the drug.
¹Approximately 100 mg (ca. 6 wt %) of water was added to each solution.
²Approximately 40 mg (ca. 3 wt %) of water was added to each solution.

The drugs shown in Table 2 represent a panel of structurally diverse drugs with a range of pKa. The HBenβCD and SulfoHBenβCD shown in Table 2 were able to solubilize this panel with relatively minimal modification of the initial stock solutions. For example, both paclitaxel (ca. molar ratio of 1:1 drug:CD) and anastrozole (ca. molar ratio of 1:3 drug:CD) were soluble in 1 g of stock solutions after mixing for ca. 48 h. The drug salts (raloxifene HCL and tamoxifen citrate) were the most resistant to solubilization with the selected stock solutions. Both drugs required the addition of 3-6 wt % (based on total weight of components) of water to achieve satisfactory solubility.

These results demonstrate that soluble pharmaceutical formulations with a diverse range of poorly water-soluble drugs can be prepared by the practice of this invention. In all cases, a relatively high drug loading was achieved.

### Example 3

### Solubility of Tamoxifen in Hydroxybutenyl Cyclodextrins:Water-Miscible Organic Solvent Mixtures

Following the procedures similar to those of example 2, tamoxifen was solubilized in selected HBenβCD_{4.7}:solvent mixtures. For every sample, the amount of material weighed was 29.4:68.6:2.1 HBenβCD_{4.7}:solvent:tamoxifen. In the case of binary solvents (Table 2), the ratio was 96.7:3.3 PEG400:cosolvent. The samples were allowed to mix until a solution with no visible particles was obtained. The samples were then analyzed by ¹H NMR. The results are summarized in Table 3.

TABLE 3
Tamoxifen (w/w drug/CD) solubilized by HBenβCD_{4.7} in
the indicated solvent system as determined by¹H NMR.

| | | Drug:HBenCD |
|---|---|---|
| Sample | Solvent | (w/w) |
| 1 | PEG 400 | 6.7 |
| 2 | PEG 400 + H₂O | 6.6 |
| 3 | PEG 400 + EtOH | 6.5 |
| 4 | PEG 400 + PG | 5.7 |

This example illustrated that hydrophobic drugs such as tamoxifen can be solubilized with hydroxybutenyl-β-cyclodextrins in the nonaqueous solvent systems of the present invention. As controls, the solubility of tamoxifen in the same solvent systems but in the absence of HBenβCD_{4.7} were also visually inspected and it was found that tamoxifen was not dissolved. That is, the HBenβCD_{4.7} serves to aid in the solubilization of tamoxifen in the pharmaceutical formulation. After storage of the solutions at ambient temperature for 5 weeks, no changes in solubility of any of the samples were observed.

### Example 4

### Release of Tamoxifen from Liquid Filled Gelatin Capsules

The tamoxifen:HBenβCD_{4.7} formulations prepared in example 3 were used to prepare liquid filled capsules. The capsules, hard shell TORPAC Lock Ring Gel Capsules (Size #0) available from Torpac Capsules Inc., Fairfield, N.J., were hand filled to 0.6 to 0.8 g of liquid tamoxifen:HBenβCD_{4.7} solutions. No degradation of the capsules were observed during storage of the capsules at ambient temperature for several days.

As a control, a dry solid inclusion complex was prepared in water and was used to fill hard shell gelatin capsules. Thus, in a 20 mL glass vial with a screw cap, dissolved 3 g of HBenβCD_{4.7} (MS=4.7) in 15 mL of unbuffered deionized water. The pH of the HBenβCD_{4.7} aqueous solution was approximately 3.3. To the HBenβCD_{4.7} aqueous solution was added 193 mg of tamoxifen. The vial was briefly vortexed then placed on a roller and the suspension was allowed to mix for approximately 8 days at ambient temperature. The pH of the tamoxifen:HBenβCD_{4.7} aqueous mixture after mixing for this period was approximately 6.5. The tamoxifen:HBenβCD_{4.7} aqueous mixture was then filtered through a 0.45 μm sterile filter into a clean flask for freeze drying. After freeze drying, 3.0268 g of a white tamoxifen:HBenβCD_{4.7} solid complex was obtained. Analysis of this tamoxifen:HBenβCD_{4.7} complex by DSC and by ¹H NMR was consistent with a tamoxifen:HBenβCD_{4.7} inclusion complex.

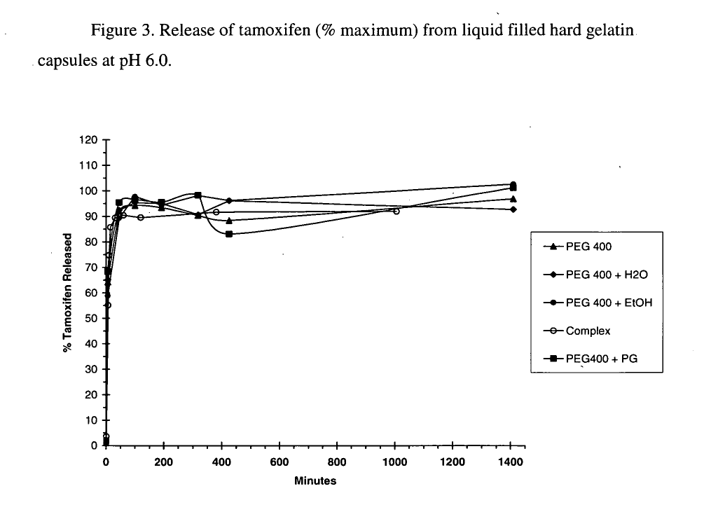

Dissolution testing was done using a USP #2 apparatus with Teflon coated paddles and 500 ml of USP pH 6.0 and 7.4 buffer solutions. For each experiment the buffer solution was heating to 42° C., followed by vacuum filtration through a 0.45 micron nylon membrane and the vacuum held for an addition 5 minutes. Buffer solutions (500 mL) were added to each of the 1000 mL glass dissolution vessels, covered and allowed to equilibrate to 37.3° C. for 30 minutes. The vessels were kept at constant temperature by a water bath kept at 37.3° C. The capsules were weighted down with a Varian 3-prong capsule weight. Once the capsules sunk to the bottom of the vessel, the test was initiated by turning the paddles at 100 rpm. The testing was done by withdrawing samples as a function of time with a 10 ml syringe. The removed samples were filtered through a 0.45 micron membrane filter, placed in scintillation vials, and immediately evaluated using a Varian UV-Vis Spectrophotometer, which had been standardized to the concentrations anticipated in the dissolution tests. The samples were measured at 275 nm with a baseline correction from 265-240 nm, using quartz absorption cells. The concentrations measured were then used to calculate the percentage of drug released from the total capsule weight. The results are summarized in FIGS. 3-6.

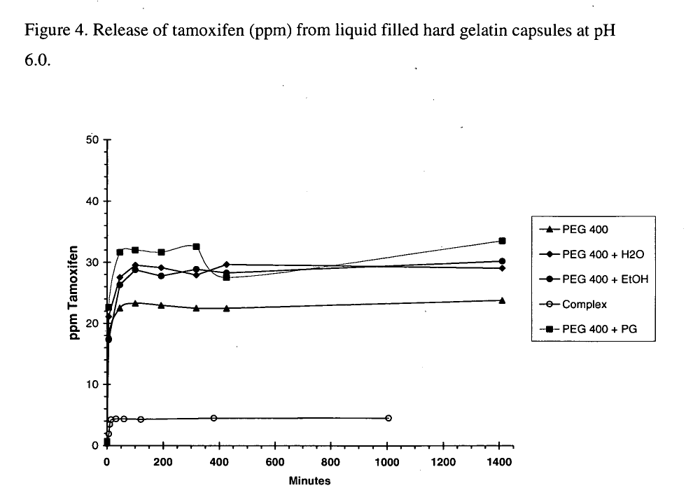

FIGS. 3 and 4 shows the dissolution and release of tamoxifen from the capsules at a pH of 6.0. For both the liquid filled capsules and solid filled capsules (FIG. 3), dissolution and release of the tamoxifen was very rapid with ca. 100% release being achieved with 45 minutes (ca. 80% at 10 minutes). No crystallization or precipitation of tamoxifen was observed during the course of the experiment (ca. 24 h). These observations demonstrate that upon exposure of the filled capsules to a simulated physiological environment, the formulations provide for rapid release and stabilization of the drug over a time period that exceeds the normal intestinal transit time for most humans (8-12 h). The liquid filled formulations provided for a higher concentration (FIG. 4) of tamoxifen (ca. 25-30 ppm) relative to the solid filled capsule (ca. 4-5 ppm). As the dry solid inclusion complex was prepared in water at equilibrium concentration, this indicates that it is possible to obtain a much higher drug loading using the pharmaceutical formulation of the present invention relative to conventional practices.

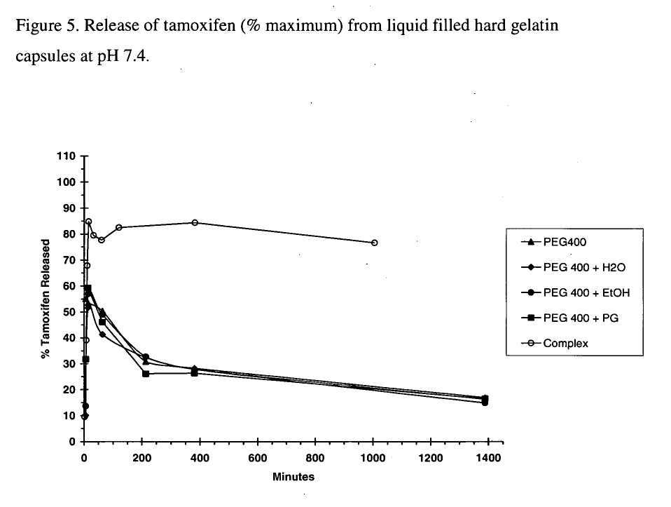

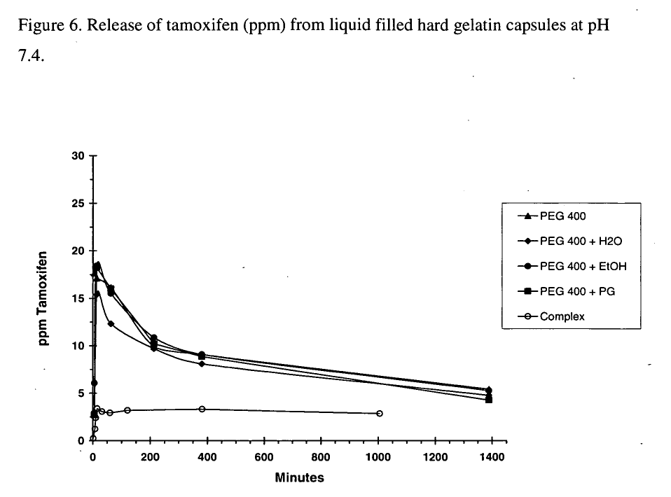

FIGS. 5 and 6 shows the dissolution and release of tamoxifen from the capsules at a pH of 7.4. At pH 7.4, the solid filled capsule provides for ca. 80% release of tamoxifen which was sustained over the course of the experiment (ca. 24 h). During the course of the experiment, tamoxifen solids were observed to form. A lower tamoxifen concentration at pH 7.4 relative to pH 6.0 is not surprising as the lowered solubility is related to the difference between the pKa of the tamoxifen base and the pH of the media. In the case of the liquid filled capsules, ca. 60% of the tamoxifen is rapidly released which then decreases to ca. 30% after 8 h. As with the solid filled capsules, during the course of the experiment tamoxifen solids were observed to form. However, relative to the solid filled capsules, the liquid filled capsules provide for a much higher concentration of tamoxifen at any point of the experiment. For example, at 8 h the liquid filled formulations provided a tamoxifen concentration of ca. 8-9 ppm versus ca. 3 ppm for the solid filled formulation. Thus at pH 7.4 the liquid filled formulations provide for a higher drug concentration relative to conventional practices.

### Example 5

### Pharmacokinetic Study Involving Administration of Tamoxifen:HBenBCD Complexes to Sprague-Dawley Rats

Solid tamoxifen base: HBenBCD and tamoxifen citrate:HBenBCD (MS=4.9) complexes were prepared in 0.05 M pH 3 phosphate buffers by the methods described in U.S. patent application entitled “Pharmaceutical Formulations of Cyclodextrins and Selective Estrogen Receptor Modulator Compounds,” filed Nov. 7, 2005, concurrently with this application (no Serial assigned at this time). The tamoxifen/HBenBCD complex contained 7.2 wt % of tamoxifen base and the tamoxifen citrate:HBenBCD complex contained 11.6 wt % tamoxifen citrate. The solid complexes were dissolved in the appropriate amount of water for aqueous oral dosing of animals. Corresponding tamoxifen base and citrate:HBenBCD complexes were prepared in a PEG400 (39.1 wt %), propylene glycol (56.5 wt %), water (4.4 wt %) solution containing 42.8 wt % HBenBCD according to the methods of Example 3. The tamoxifen/HBenBCD solution contained 0.62 wt % of tamoxifen base and the tamoxifen citrate:HBenBCD complex contained 2.46 wt % tamoxifen citrate.

A summary of the study design is provided in Table 4. The study animal were male Sprague-Dawley rats. The study was broken into four main groups that were to receive oral doses of aqueous tamoxifen base:HBenBCD (Group 1), aqueous tamoxifen citrate:HBenBCD solutions (Group 2), PEG400/PG tamoxifen base:HBenBCD solutions (Group 3), and PEG400/PG tamoxifen citrate:HBenBCD solutions (Group 4) by gavage. Each group was sub-divided into 4 groups of 3 animals on the basis of time of dosing (AM or PM) and diet (fed or fasted). In the case of fasted animals, food was removed 8 hours prior to dosing then returned 5 hours post-dose. The rats in each group were to be given a single dose equivalent to 10 mg/Kg of tamoxifen base.

TABLE 4
Study design for administration of single oral doses of
tamoxifen:HBenBCD complexes to Sprague Dawley rats.

| | Group | | Dietary | Average Dose |
|---|---|---|---|---|
| Test Article | (n = 3) | Dosing Time | Status | (mg/Kg)^{(a)} |
| Aqueous Oral | 1 | 9:20 AM | Fed | 10.0 |
| Solution | 2 | 13:02 PM | Fed | 10.0 |
| HBenBCD/ | 3 | 9:05 AM | Fasted | 10.0 |
| Tamoxifen Base | 4 | 13:04 PM | Fasted | 10.0 |
| Aqueous Oral | 5 | 9:32 AM | Fed | 9.9 |
| Solution | 6 | 13:14 PM | Fed | 10.0 |
| HBenBCD/ | 7 | 9:17 AM | Fasted | 10.0 |
| Tamoxifen Citrate | 8 | 13:16 PM | Fasted | 10.0 |
| PEG400 Oral | 9 | 9:06 AM | Fed | 9.7 |
| Solution | 10 | 13:26 PM | Fed | 9.5 |
| HBenBCD/ | 11 | 9:29 AM | Fasted | 9.9 |
| Tamoxifen Base | 12 | 13:34 PM | Fasted | 9.1^{(b)} |
| PEG400 Oral | 13 | 9:21 AM | Fed | 10.1 |
| Solution HBenBCD/ | 14 | 13:38 PM | Fed | 9.8 |
| Tamoxifen Citrate | 15 | 9:33 AM | Fasted | 9.9 |
| | 16 | 13:30 PM | Fasted | 10.0 |

^{(a)}Actual dose given based on tamoxifen base. The value given is the average value for the group (n = 3).
^{(b)}n = 1. Limited sample.

Blood samples (ca. 300 μL) were collected from each animal via the jugular or sublingual vein at 1, 2, 3, 4, 6, 12 and 24 hours post-dose. Plasma was separated and stored in 96 well plates at −17 to −80° C. until analysis. The concentration of tamoxifen, hydroxytamoxifen, and desmethyltamoxifen in the plasma were determined using LC-MS-MS spectrometry. Plasma (100 μL) was precipitated with 250 μL of acetonitrile. After centrifugation, the supernatant was analyzed for tamoxifen, hydroxytamoxifen, and desmethyltamoxifen by reversed phase LC-MS-MS using diphenhydramine as an internal standard.

For the purposes of determining oral bioavailability, two additional groups (n=12) received a single intravenous dose of 10 mg/Kg tamoxifen administered as aqueous tamoxifen base:HBenBCD or tamoxifen citrate:HBenBCD complexes. A third group (n=6) received a single 10 mg/Kg oral dose of a tamoxifen base aqueous suspension. This oral suspension dose was given to fed animals in the AM.

The area under the plasma concentration-time curve (AUC) from time zero to 24 h (AUC₀₋₂₄ₕ) and from time zero to infinity (AUC_{0-∞}) was calculated by standard methods (Clinical Pharmacokinetics, Malcolm Rowland and Thomas Tozer, Lippincott, Williams, & Wilkins, 1995). Oral bioavailability was determined using the AUC from the IV and oral doses adjusted for the animal dose.

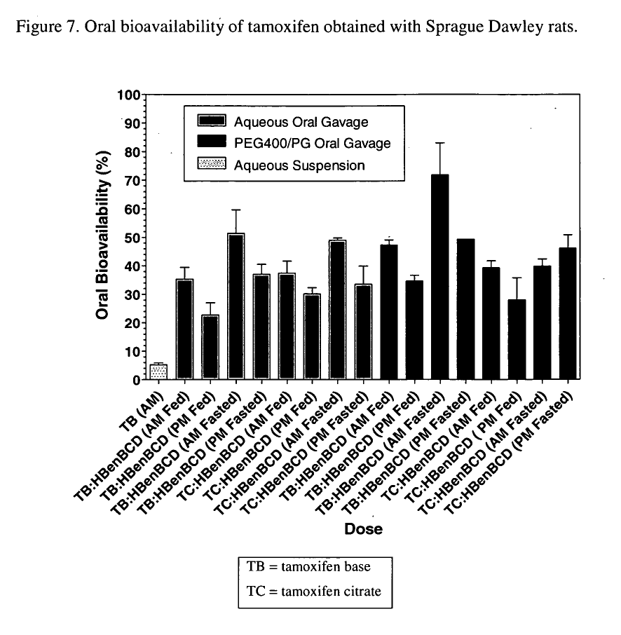

FIG. 7 shows the oral bioavailability of tamoxifen obtained from this study. When the animals were dosed with an oral suspension of tamoxifen base (no HBenBCD), the observed oral bioavailability was only 5%. The oral bioavailability for all of the tamoxifen:HBenBCD treatment groups ranged from 28 to 74% depending upon the study group. This represents a 6-15× increase in oral bioavailability of tamoxifen when tamoxifen is administered as complex with HBenBCD.

Statistical analysis of the tamoxifen:HBenBCD treatment groups revealed that there was not a significant difference in oral bioavailability when the complex was prepared using tamoxifen base versus tamoxifen citrate regardless of the dietary state or the time of dosing. This analysis shows that the extra manufacturing step to form the salt of tamoxifen is not required when tamoxifen is given as a complex with HBenBCD. For statistical analysis purposes, this observation also allowed pooling of the base-citrate groups effectively doubling the size of each study group (n=3→n=6).

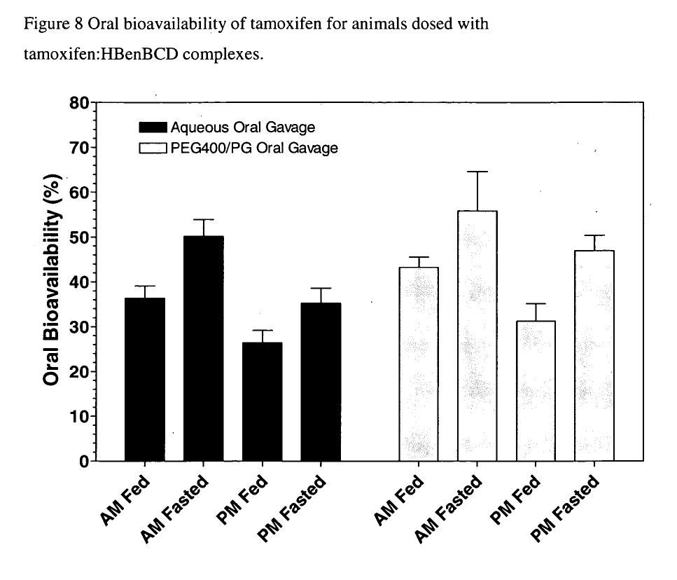

FIG. 8 compares the oral bioavailability obtained with groups dosed with tamoxifen:HBenBCD by aqueous oral solution gavage versus PEG400/PG oral solution gavage; the tamoxifen base-tamoxifen citrate treatment groups have been pooled on the basis of time of dosing-dietary status. As can be seen from FIG. 8, in both groups, both the time of the dose and dietary status influence tamoxifen oral bioavailability. Oral bioavailability is the highest in animals that are fasted and dosed in the morning in both oral gavage groups. Statistical analysis indicated that both dietary status and dosing time were significant (P<0.05) and that there was no interaction between these terms. Comparing the two oral gavage groups dosed at the same time with the same dietary status, it can be seen that in every treatment group, the greatest oral bioavailability was obtained when the dose was administered as a PEG400/PG oral solution (P<0.05).

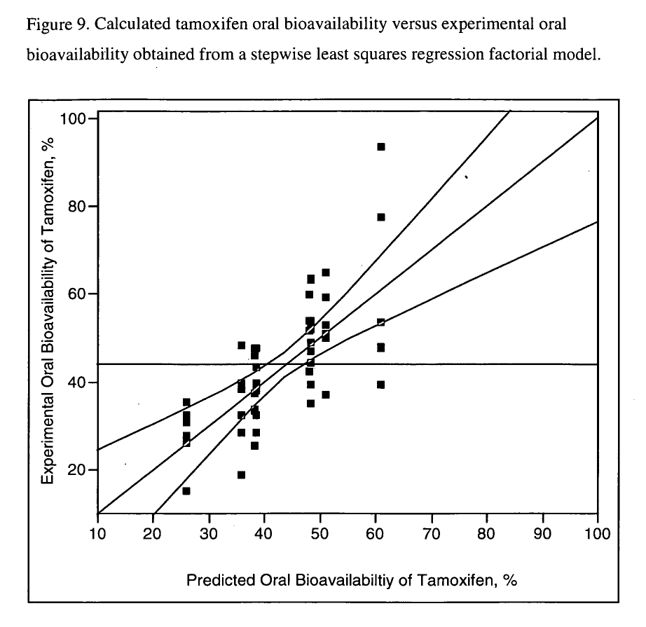

To further clarify these observations, a stepwise least squares regression factorial model was constructed in which the form of tamoxifen (base versus citrate) in the complex, time of dosing (AM vs PM), dietary status (fed versus fasted), and gavage solution (aqueous versus PEG400/PG) where allowed to independently vary. The model converged when the calculated tamoxifen oral bioavailabilites gave the best fit to the experimental oral bioavailability. Variables with P>0.05 were not considered significant and were excluded from the model. This model is illustrated in FIG. 9 which shows a plot of calculated tamoxifen oral bioavailability versus experimental oral bioavailability. The model was highly significant (P<0.0001) and 50% of the variability in the oral bioavailability was accounted for in the model. The forms of tamoxifen and interaction terms were not significant and were excluded. The model showed that oral bioavailability of tamoxifen administered as HBenBCD complexes increased when the gavage solution was PEG400/PG (P<0.0014), when the animals were dosed in the morning (P<0.0001), and when the animals were fasted prior to dosing (P<0.0001).

Administration of tamoxifen as a tamoxifen:HBenBCD pharmaceutical formulation dramatically increases the oral bioavailability of tamoxifen (6-15×) relative to dosing tamoxifen base in the absence of HBenBCD. The formulations of the present invention provided tamoxifen oral bioavailability that was superior to corresponding aqueous pharmaceutical formulations. The chronological and food effects on the oral bioavailability of tamoxifen are significant and unexpected.

It should be understood that the foregoing relates only to preferred embodiments of the present invention and that numerous modifications or alterations can be made therein without departing from the spirit and the scope of the present invention as defined in the following claims.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with a true scope and spirit of the invention being indicated by the following claims.

What is claimed is:

1. A composition comprising a water-miscible organic solvent, a cyclodextrin derivative and a compound, wherein the composition contains 10 wt % or less water.

2. The composition of claim 1, wherein the composition contains 5 wt % or less water.

3. The composition of claim 1, wherein the composition contains 2 wt % or less water.

4. The composition of claim 1, wherein the cyclodextrin derivative comprises a hydroxybutenyl cyclodextrin.

5. The composition of claim 4, wherein the hydroxybutenyl cyclodextrin comprises hydroxybutenyl-β-cyclodextrin.

6. The composition of claim 4, wherein the hydroxybutenyl cyclodextrin comprises a sulfonated hydroxybutenyl cyclodextrin.

7. The composition of claim 6, wherein the sulfonated hydroxybutenyl cyclodextrin comprises sulfonated hydroxybutenyl-β-cyclodextrin.

8. The composition of claim 1, wherein the water-miscible organic solvent comprises a natural polymer, a synthetic polymer, a semisynthetic polymer, an alcohol, a polyol, or a mixture thereof.

9. The composition of claim 1, wherein the water-miscible organic solvent comprises polyethylene glycol, ethanol, propylene glycol, glycerin, or a mixture thereof.

10. The composition of claim 1, wherein the compound comprises a pharmaceutically active compound or salt, structural analog or metabolite thereof.

11. A composition comprising a water-miscible organic solvent, a cyclodextrin derivative and a compound, wherein the water-miscible organic solvent contains 10 wt % or less water.

12. The composition of claim 11, wherein the water-miscible organic solvent contains 5 wt % or less water.

13. The composition of claim 11, wherein the water-miscible organic solvent contains 2 wt % or less water.

14. The composition of claim 11, wherein the cyclodextrin derivative comprises a hydroxybutenyl cyclodextrin.

15. The composition of claim 14, wherein the hydroxybutenyl cyclodextrin comprises hydroxybutenyl-β-cyclodextrin.

16. The composition of claim 14, wherein the hydroxybutenyl cyclodextrin comprises a sulfonated hydroxybutenyl cyclodextrin.

17. The composition of claim 16, wherein the sulfonated hydroxybutenyl cyclodextrin comprises sulfonated hydroxybutenyl-β-cyclodextrin.

18. The composition of claim 11, wherein the water-miscible organic solvent comprises a natural polymer, a synthetic polymer, a semisynthetic polymer, an alcohol, a polyol, or a mixture thereof.

19. The composition of claim 11, wherein the water-miscible organic solvent comprises polyethylene glycol, ethanol, propylene glycol, glycerin, or a mixture thereof.

20. The composition of claim 11, wherein the compound comprises a pharmaceutically active compound or salt, structural analog or metabolite thereof.

21. A method of enhancing the solubility of a compound in aqueous media comprising forming a mixture or complex between the compound and a cyclodextrin derivative in a water-miscible organic solvent, wherein the mixture or complex contains 10 wt % or less water.

22. A method of increasing the bioavailability of a compound in a subject comprising administering the composition of claim 1 to the subject.

23. The method of claim 22, wherein the subject is a human.

24. A method of increasing the bioavailability of a compound in a subject comprising administering the composition of claim 11 to the subject.

25. The method of claim 24, wherein the subject is a human.
